(19) United States
(12) Patent Application Publication
(10) Pub. No.: US 2011/0160853 A1
(43) Pub. Date: Jun. 30, 2011

(54) CAPSULAR TENSION RING WITH COIL FOR INDUCTIVE COUPLING TO AN EXTERNAL ELECTROMAGNETIC FIELD

(76) Inventor: Dick Scholten, Stuttgart (DE)

(21) Appl. No.: 12/737,189

(22) PCT Filed: Jun. 26, 2009
(86) PCT No.: PCT/EP2009/058011

§ 371 Date: Mar. 2, 2011

(30) Foreign Application Priority Data
Jul. 3, 2008 (DE) 10 2008 040 142.0

(51) Int. Cl. A61F 2/16; H01F 38/14

(52) U.S. Cl. 623/6.43; 307/104

(57) ABSTRACT

A closed capsular tension ring, with a coil having at least one winding turn 6, wherein the winding turn 6 is disposed in the capsular tension ring 1. The closed capsular tension ring according to the invention has the advantage that it may be used without limitation to a particular artificial lens or to a particular shape of artificial lens.

### BACKGROUND OF THE INVENTION

1. Field of the Invention

The present invention relates to a closed capsular tension ring.

Capsular tension rings are used to stabilize the capsular bag in the eye and are inserted into the capsular bag as an implant, for example after removal of the natural lens of an eye, to support the capsular tissue. After removal of the natural lens, for example because of severe opacification, it is necessary that the opened capsular bag remains substantially in its original shape and is thereby able to facilitate the implantation of an artificial intraocular lens.

As a consequence of an increased mean intraocular pressure, for example in the case of the eye disorder glaucoma, the nerve fibers of the retina die off. In many cases it is possible for the intraocular pressure to be reduced to an acceptable value by eye drop medication. For optimum long-term therapy, however, the dosage of the eye drop medication has to be adapted to the mean intraocular pressure. Although it is possible for the intraocular pressure to be measured relatively accurately by an ophthalmologist, sporadic measurement is inadequate in many cases since the intraocular pressure depends among other things on the factors body position, time of day and/or stress.

2. Description of Related Art

Published German patent document DE 197 28 069 C1 describes integration of a pressure sensor for measuring intraocular pressure in an artificial lens. The device described has a telemetry system including a pressure sensor, a data logger which temporarily stores the sensor signals, a device which converts the sensor signals into wirelessly transmissible information, and a transmitting device. A receiving device disposed outside the eye is also described, which receives from the transmitting device the information retrieved from the data logger over a limited period of time and which is connected to an evaluation device in which the received information is converted into intraocular pressure data for charting purposes.

That construction and also other constructions known from the related art allow only a very restricted selection of possible artificial lenses into which such a pressure sensor may be integrated to measure intraocular pressure.

### SUMMARY OF THE INVENTION

The closed capsular tension ring in accordance with the present invention has, by comparison, the advantage that it may be used without limitation to a particular artificial lens or to a particular shape of artificial lens.

In accordance with the present invention, that is achieved by a closed capsular tension ring with a coil having at least one winding turn, wherein the winding turn is disposed in the capsular tension ring.

In accordance with the present invention, therefore, a closed capsular tension ring, which is required, for example, after a cataract operation, is provided. With the use of a minimally invasive implantation procedure, the capsular bag in the eye may be kept in shape by the closed capsular tension according to the invention. In addition to the capsular tension ring, any desired artificial lens may be inserted into the eye.

A preferred development of the present invention further provides that the closed capsular tension ring is formed from at least one flexible segment and at least one rigid segment, wherein a flexible segment and a rigid segment are arranged in alternating sequence in each case.

A preferred development of the present invention provides that the capsular tension ring has a device for measuring and/or for controlling a physical quantity, wherein the coil has a connection to the unit. A further preferred development of the present invention provides that the device is in the form of a pressure measuring unit for measuring the intraocular pressure. Suitable pressure measuring units are sensors with which intraocular pressure may be detected and also other sensors known from the related art for insertion into blood vessels and as cardiac catheters, which are preferably constructed using surface-micromachining technology. The provision of a closed capsular tension ring makes it possible to integrate a coil having one or more winding turns which are connected to the pressure measuring unit. That makes it possible to measure the intraocular pressure without being limited to a particular shape of artificial lens. Provision may also be made for other physical quantities to be measured by the unit, such as temperature or chemical substances. A medicament may also be administered in metered doses by the unit.

A preferred development of the present invention further consists in the flexible segment being foldable and/or bendable. That makes it possible for the capsular tension ring to be folded together. More particularly, a preferred development of the present invention provides that the capsular tension ring may be folded together along a fold line. In addition, it is preferred that the capsular tension ring may be folded together especially along the equatorial line and may therefore have a semicircular-type shape in the folded-together state. In that folded-together shape, the capsular tension ring may be implanted into the eye in an especially simple manner by a minimally invasive implantation procedure. In other words, the capsular tension ring may be “screwed” into the eye through an opening by such a minimally invasive implantation procedure.

In addition, it is preferred that the coil is provided for supplying power to the unit and/or for data transmission of the unit and/or for data transmission of the physical quantity measured by the unit. Preferably, the power is supplied and/or the data are transmitted inductively.

The unit may in principle be disposed at any desired position on/in the capsular tension ring. A preferred development of the invention provides, however, that the unit is disposed on/in the flexible segment. For example, the unit may be encapsulated by the application of silicone.

A preferred development of the present invention provides that two flexible segments and two rigid segments are provided. In addition, it is preferred that the length of the rigid segment is greater than the length of the flexible segment. Preferably, the length of the flexible segment is such that that it permits the capsular tension ring to be folded together, while the length of the rigid segment makes up the majority of the circumference of the capsular tension ring. In accordance with this development of the invention, that is to say in the case of two rigid segments, the length of the rigid segment is preferably substantially half the circumference of the capsular tension ring. In that manner, a high stability of the capsular tension ring may be achieved.

In addition, a preferred development of the present invention provides that three rigid segments are provided, wherein the length of the first rigid segment is greater than the sum of the lengths of the second rigid segment and the third rigid segment. Particularly preferably, the length of the first rigid segment substantially corresponds to half the circumference of the capsular tension ring, and the lengths of the second rigid segment and the third rigid segment each correspond substantially to one quarter of the circumference of the capsular tension ring.

As has been said above, the unit may be disposed at any desired position on/in the capsular tension ring. A preferred development of the invention provides, however, that three flexible segments are provided, wherein the unit is disposed on/in that flexible segment which is disposed between the second rigid segment and the third rigid segment. Accordingly, the capsular tension ring may be folded together along the fold line, which preferably corresponds substantially to the equator of the capsular tension ring, without the folding of the capsular tension ring involving the flexible segment in which the pressure measuring unit is disposed. A folded-together capsular tension ring of that kind may be implanted into the eye by a minimally invasive implantation procedure, with the capsular tension ring preferably unfolding after implantation in the eye.

A preferred development further consists in the rigid segment being made from polymethylmethacrylate (PMMA). Preferably, the flexible segment is made from hydroxyethylmethacrylate-co-methylmethacrylate (HEMA/MMA) or silicone. Furthermore, the flexible segment may also have hydrophilic properties. That improves the foldability and/or bendability of the capsular tension ring, thereby facilitating implantation of the capsular tension ring into the opened capsular bag.

The present invention further relates to an apparatus for measuring the intraocular pressure, having a closed capsular tension ring according to the invention and an external device, provided outside the eye, for supplying power to the coil and for data transmission with the coil. In other words, the external device is able to supply the coil of the closed capsular tension ring with power transmitted inductively, so that, for example, the pressure measuring unit connected to the coil is able to measure the intraocular pressure. The intraocular pressure measured by the pressure measuring unit may then be transmitted inductively to the external device via the coil. The inductively transmitted data of the pressure measuring unit may be stored, analyzed and/or visualized on a data evaluation device.

The above-described closed capsular tension ring and/or the apparatus for measuring the intraocular pressure are preferably used in medical technology, for example in the case of the eye disorder glaucoma already mentioned. Further areas of use are in intraocular pressure measurement for other medical applications.

### BRIEF DESCRIPTION OF THE DRAWINGS

The invention is described in detail below using preferred exemplary embodiments and referring to the drawings. In the drawings:

FIG. 1 is a schematic cross-sectional view of a closed capsular tension ring in accordance with a preferred exemplary embodiment of the invention.

FIG. 2 is a schematic cross-sectional view of the closed capsular tension ring in accordance with a further preferred exemplary embodiment of the invention.

### DETAILED DESCRIPTION OF THE INVENTION

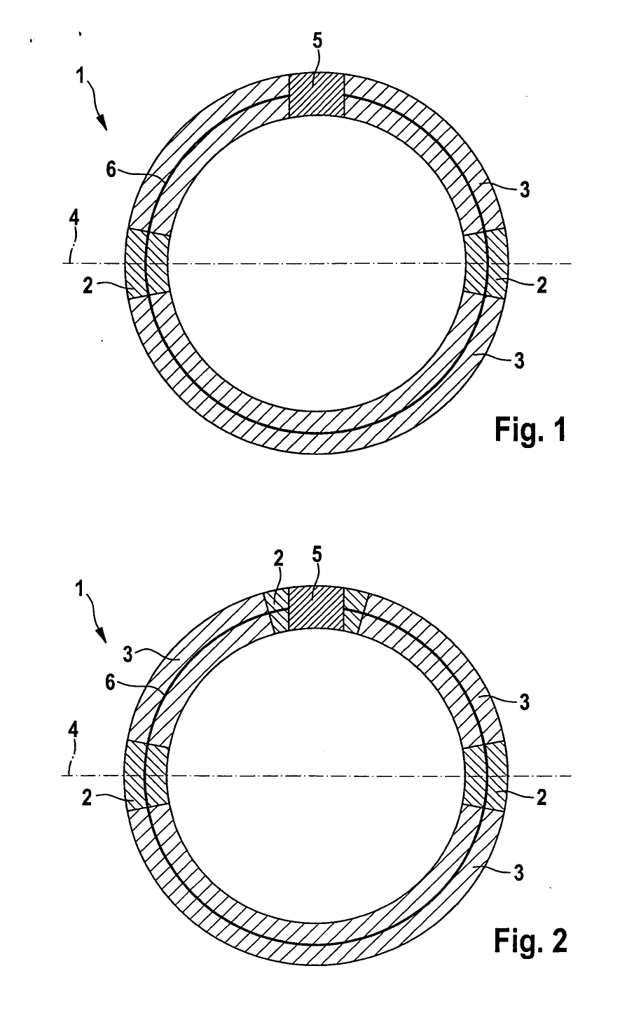

FIGS. 1 and 2 each show a schematic cross-sectional view of a closed capsular tension ring 1 in accordance with a preferred exemplary embodiment of the present invention. Capsular tension ring 1 shown in FIG. 1 has two flexible segments 2 and two rigid segments 3, a flexible segment 2 and a rigid segment 3 being arranged in alternating sequence in each case.

In accordance with the preferred exemplary embodiment of the invention, flexible segments 2 are of a foldable and/or bendable configuration, so that capsular tension ring 1 may be folded together along a fold line 4. A capsular tension ring 1 folded together in that manner may be inserted into an eye through an opening by a minimally invasive implantation procedure. Preferably, capsular tension ring 1 unfolds after implantation in the eye.

Flexible segments 2 and rigid segments 3 may in principle be made from any desired materials. In accordance with the preferred exemplary embodiment of the invention, it is preferred, however, that flexible segment 2 is made from hydroxyethylmethacrylate-co-methylmethacrylate (HEMA/MMA) or silicone. Rigid segment 3 is preferably made from polymethyl-methacrylate (PMMA).

As may be seen from FIG. 1, the length of rigid segment 3 substantially corresponds to half the circumference of capsular tension ring 1. In that manner, a high stability of capsular tension ring 1 is achieved.

The closed capsular tension ring also has a unit 5 which, as may be seen from FIG. 1, is disposed in rigid segment 3 of capsular tension ring 1 and, in accordance with this exemplary embodiment of the invention, is in the form of a pressure measuring unit. Using the pressure measuring unit it is possible to measure the intraocular pressure. In accordance with this exemplary embodiment of the invention, a coil (not shown), which is connected to the pressure measuring device and has at least one winding turn 6, is provided. The coil serves to supply power to the pressure measuring unit and for data transmission of the intraocular pressure measured by the pressure measuring unit. Power is supplied preferably inductively by an external device, the external device also being provided for inductive data transmission with the coil.

In accordance with a further preferred exemplary embodiment of the present invention, the closed capsular tension ring has three flexible segments 2 and three rigid segments 3, as may be seen from FIG. 2. In accordance with this preferred exemplary embodiment of the invention, the pressure measuring unit is provided in flexible segment 2. Preferably, the length of first rigid segment 3 substantially corresponds to half the circumference of capsular tension ring 1, while the length of second rigid segment 3 and of third rigid segment 3 substantially corresponds to the length of one quarter of the circumference of capsular tension ring 1. Accordingly, fold line 4 also corresponds substantially to the equator of capsular tension ring 1.

As may further be seen from FIG. 2, the pressure measuring unit is provided in that flexible segment 2 which is disposed between the two rigid segments 3 that substantially correspond in their length to one quarter of capsular tension ring 1.

1-14. (canceled)

15. A closed capsular tension ring, comprising a coil having at least one winding turn, wherein the winding turn is disposed in the capsular tension ring.

16. The closed capsular tension ring as recited in claim 15, wherein the ring has at least one flexible segment and at least one rigid segment arranged in alternating sequence in each case.

17. The closed capsular tension ring as recited in claim 15, further comprising a device for measuring or for controlling a physical quantity, wherein the coil has a connection to the device.

18. The closed capsular tension ring as recited in claim 17, wherein the device is a pressure measuring unit for measuring intraocular pressure.

19. The closed capsular tension ring as recited in claim 15, wherein the flexible segment is of a foldable or bendable configuration.

20. The closed capsular tension ring as recited in claim 15, wherein the capsular tension ring may be folded together along a fold line.

21. The closed capsular tension ring as recited in claim 15, wherein the coil is provided for at least one of: supplying power to the device; data transmission of the device; and data transmission of the physical quantity measured by the device.

22. The closed capsular tension ring as recited in claim 17, wherein the ring has at least one flexible segment and wherein the device is disposed on or in the flexible segment.

23. The closed capsular tension ring as recited in claim 16, wherein two flexible segments and two rigid segments are provided.

24. The closed capsular tension ring as recited in claim 16, wherein the length of the rigid segment is greater than the length of the flexible segment.

25. The closed capsular tension ring as recited in claim 15, wherein the ring has three rigid segments, and wherein the length of the first rigid segment is greater than the sum of the lengths of the second rigid segment and the third rigid segment.

26. The closed capsular tension ring as recited in claim 25, wherein the ring has three flexible segments and wherein one of the flexible segments is disposed between the second rigid segment and the third rigid segment.

27. The closed capsular tension ring as recited in claim 26, further comprising a device for measuring or for controlling a physical quantity, the coil having a connection to the device, and the device being disposed on the flexible segment disposed between the second rigid segment and the third rigid segment.

28. The closed capsular tension ring as recited in claim 15, wherein the rigid segment is made from polymethylmethacrylate.

29. An apparatus for measuring intraocular pressure comprising a closed capsular tension ring as recited in claim 15 and an external device, the external device being disposed outside the eye for supplying power to the coil and for data transmission with the coil.
